(19) United States
(12) Patent Application Publication
(10) Pub. No.: US 2022/0223241 A1
(43) Pub. Date: Jul. 14, 2022

(54) METHODS AND SYSTEMS FOR GENERATING PERSONALIZED RECOMMENDATIONS AND PREDICTIONS OF A LEVEL OF EFFECTIVENESS OF THE PERSONALIZED RECOMMENDATIONS FOR A USER

(71) Applicant: juli, Inc., Hull, MA (US)

(72) Inventors: Joris Gijsbert Straatman, Zürich (CH); Joseph Hayes, London (GB); Bettina Dührkoop, Küsnacht (CH); Bettina Hein, St. Gallen (CH)

(21) Appl. No.: 17/562,313

(22) Filed: Dec. 27, 2021

Related U.S. Application Data
(60) Provisional application No. 63/135,856, filed on Jan. 11, 2021.

(51) Int. Cl. G16H 10/60; G16H 20/30

(52) U.S. Cl. CPC G16H 10/60; G16H 20/30

(57) ABSTRACT

A method for generating at least one personalized recommendation and a prediction of a level of effectiveness of the personalized recommendation for a type of user includes receiving, by an application executing on a computing device, via a user interface, user input identifying a portion of a quadrant displayed within the user interface descriptive of an emotional state of a user and of an energetic state of a user. The application accesses, from at least one data source, data associated with the user. The application analyzes the accessed data and the received user input. The application identifies a user health pattern. The application determines whether the health pattern is aligned with a user goal. The application generates a recommendation to the user to align at least one user behavior pattern with the user goal, the recommendations personalized to a preference of the user and satisfying a threshold level of effectiveness.

### CROSS-REFERENCE TO RELATED APPLICATIONS

This application claims priority from U.S. Provisional Patent Application No. 63/135,856, filed on Jan. 11, 2021, entitled, “Methods and Systems for Generating Personalized Recommendations and Predictions of a Level of Effectiveness of the Personalized Recommendations for a User,” which is hereby incorporated by reference.

### BACKGROUND

Although conventional systems for tracking health issues and generating health recommendations for users exist, such systems do not conventionally provide customized recommendations for users based both on users' health-related data inputs or measures of wellbeing (including, e.g., levels of energy and emotional state), and based on a likelihood the user will effectively implement the recommendations. There is a need for identifying personalized improvement strategies and behavioral chances for users with complex chronic health conditions, often occurring in combinations, optimized by learning personal preferences of users, identified by accessing many data sources and improved by learning from a user base. This is in contrast to conventional solutions, which typically focus only on one condition instead of combinations of conditions, present generic strategies instead of personalized strategies, and/or do not typically integrate data from electronic health records (e.g., EHR/FHIR data).

### BRIEF SUMMARY

In one aspect, a method for generating at least one personalized recommendation and a prediction of a level of effectiveness of the personalized recommendation for a type of user includes receiving, by an application executing on a computing device, via a user interface, user input identifying a portion of a quadrant displayed within the user interface descriptive of an emotional state of a user and of an energetic state of a user. The method includes accessing, by the application, from at least one data source, data associated with the user. The method includes analyzing, by the application, the accessed data and the received user input. The method includes identifying, by the application, a health pattern of the user based on the analyses. The method includes determining, by the application, whether the health pattern of the user is aligned with a goal of the user. The method includes generating, by the application, a recommendation to the user to align at least one user behavior pattern with the goal of the user, the recommendations personalized to a preference of the user and having a level of effectiveness satisfying a threshold level of effectiveness. The method includes modifying, by the application, the user interface to display the recommendation.

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIG. 1 is a flow diagram depicting an embodiment of a method for generating personalized recommendations and predictions of a level of effectiveness of the personalized recommendations for a type of user;

FIG. 2A is a block diagram depicting an embodiment of a system for generating personalized recommendations and predictions of a level of effectiveness of the personalized recommendations for a type of user;

FIG. 2B is a block diagram depicting an embodiment of a user interface embodiment of a plurality of Application Programming Interfaces (APIs) provided in a system for generating personalized recommendations and predictions of a level of effectiveness of the personalized recommendations for a type of user;

FIG. 2C is a block diagram depicting an embodiment of a plurality of services provided in a system for generating personalized recommendations and predictions of a level of effectiveness of the personalized recommendations for a type of user;

FIG. 2D is a block diagram depicting an embodiment of a plurality of engines executed in a system for generating personalized recommendations and predictions of a level of effectiveness of the personalized recommendations for a type of user;

FIG. 2E is a block diagram depicting an embodiment of a plurality of user-focused services and a plurality of data science engines executed in a system for generating personalized recommendations and predictions of a level of effectiveness of the personalized recommendations for a type of user;

FIG. 2F is a block diagram depicting an embodiment of a plurality of engines executed in a system for generating personalized recommendations and predictions of a level of effectiveness of the personalized recommendations for a type of user; and

FIGS. 3A-3C are block diagrams depicting embodiments of computers useful in connection with the methods and systems described herein.

### DETAILED DESCRIPTION

In one aspect, the methods and systems described herein provide functionality for identifying a level of overall well-being of a user related to his/her condition(s) through both user-reported data and passively sensed elements of the user's health; such methods and systems may include functionality providing users with recommendations for improving and/or stabilizing their state of well-being.

In one aspect, the methods and systems described herein provide functionality for receiving user input identifying user-reported state of emotion and user-reported level of energy and providing users with recommendations for improving and/or stabilizing their emotional states and levels of energy; such recommendations may be personalized to match both a condition of the user and a level of effectiveness of the recommendation for the user (e.g., how likely the user is to effectively implement the recommendation). The methods and systems described herein may include functionality for analyzing the received user input, determining a pattern associated with the received user input, identifying a position along a graph based upon the pattern that represents not just the user's present state but also a prediction of a future state the user will experience, and making a recommendation for a behavioral modification that the user is likely to implement effectively and that is likely to further a user's progress towards a goal (e.g., not sliding into depression, not gaining or losing an amount of weight in excess of a user-specified threshold, increasing or decreasing levels of activity or socialization, and so on).

In another aspect, the methods and systems described herein provide functionality for receiving user input identifying user-reported state of well-being and providing users with recommendations for improving and/or stabilizing a condition such as asthma. In one such embodiment, users provide input representing a level of wellbeing, including data relating to incidents of shortness of breath, use of an emergency inhaler, wakefulness due to shortness of breath, and so on.

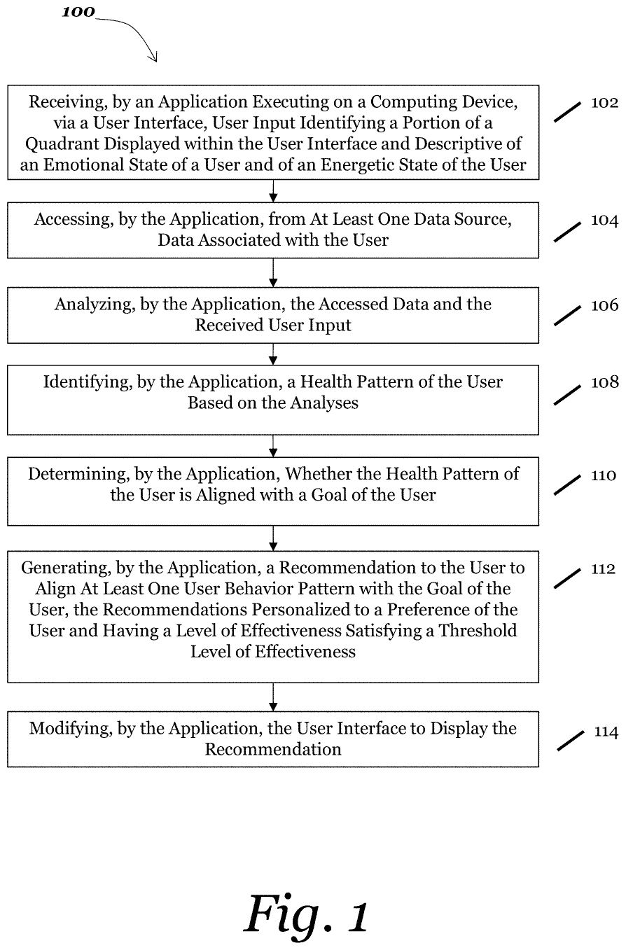

Referring now to FIG. 1, in brief overview, a flow diagram depicts an embodiment of a method 100 for generating at least one personalized recommendation and a prediction of a level of effectiveness of the personalized recommendation for a type of user. The method includes receiving, by an application executing on a computing device, via a user interface, user input identifying a portion of a quadrant displayed within the user interface and descriptive of an emotional state of a user and of an energetic state of the user (102). The method includes accessing, by the application, from at least one data source, data associated with the user (104). The method includes analyzing, by the application, the accessed data and the received user input (106). The method includes identifying, by the application, a health pattern of the user based on the analyses (108). The method includes determining, by the application, whether the health pattern of the user is aligned with a goal of the user (110). The method includes generating, by the application, a recommendation to the user to align at least one user behavior pattern with the goal of the user, the recommendations personalized to a preference of the user and having a level of effectiveness satisfying a threshold level of effectiveness (112). The method includes modifying, by the application, the user interface to display the recommendation (114). In one embodiment, therefore, the methods and systems described herein provide a platform with which users may interact through software applications that gather and store data and which provide users with feedback including, without limitation, advice, reports, and alerts.

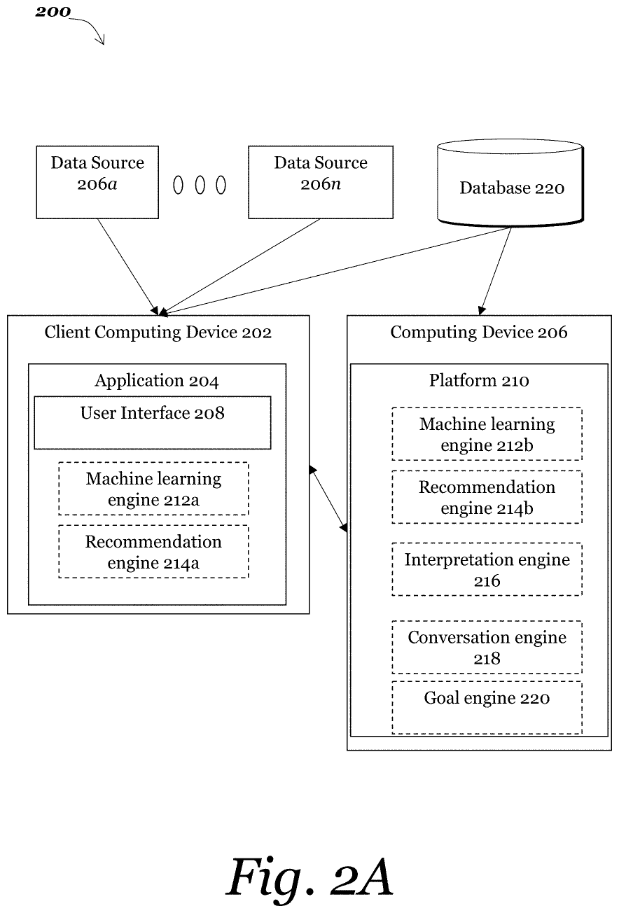

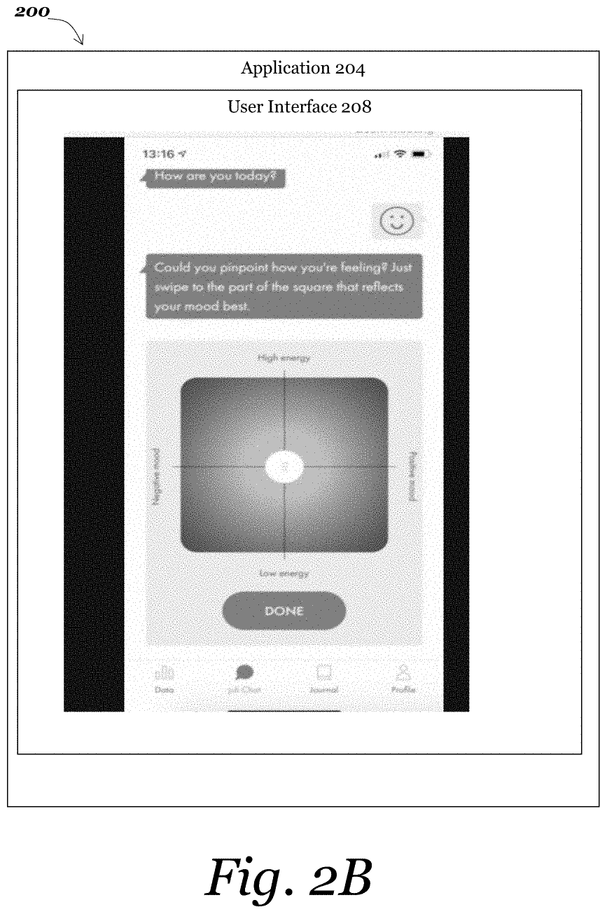

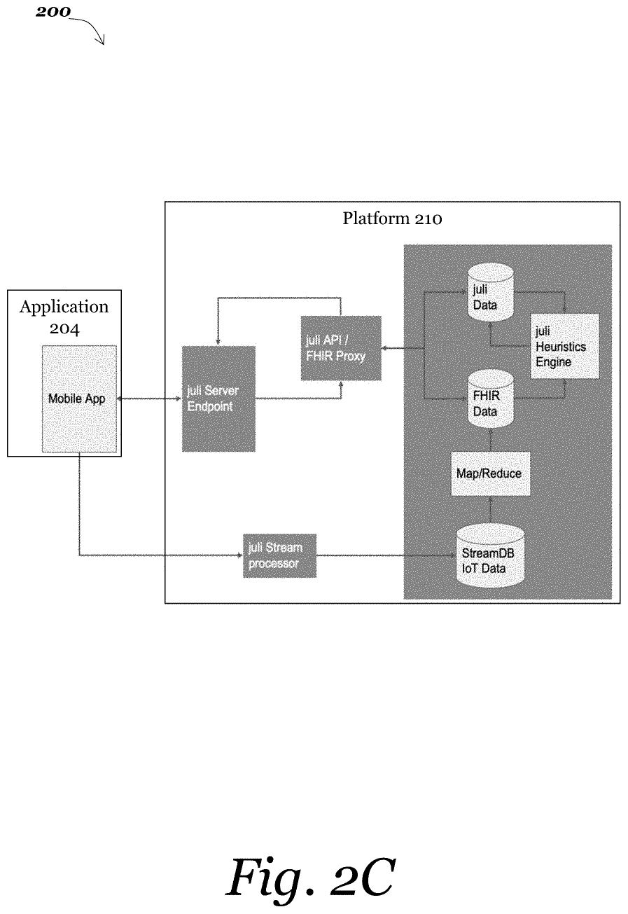

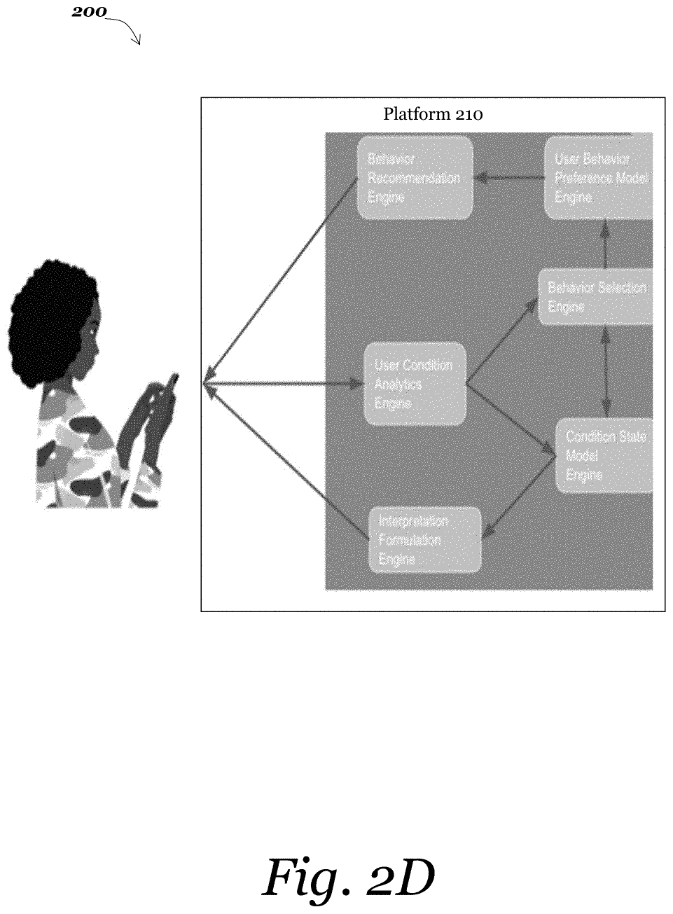

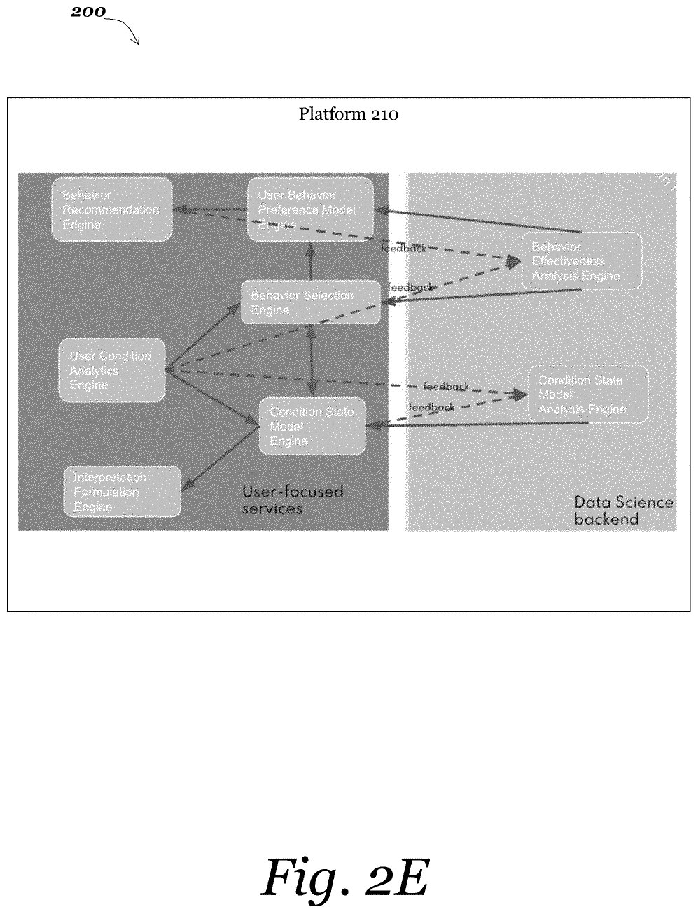

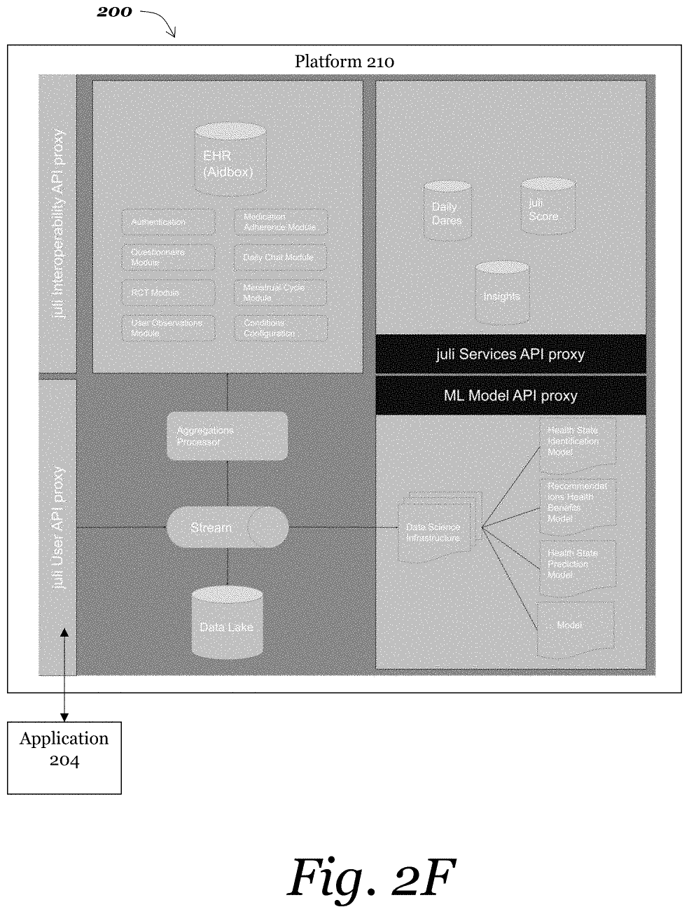

Referring now to FIG. 1 in greater detail, and in connection with FIG. 2A-B, the method includes receiving, by an application executing on a computing device, via a user interface, user input identifying a portion of a quadrant displayed within the user interface descriptive of an emotional state of a user and of an energetic state of a user (102). The application 204 may execute on a client computing device 202 such as, without limitation, a user's phone or personal computing device. As shown in FIG. 2B, the application 204 may generate and display a user interface 208 with which a user may provide user input.

Chronic conditions, such as bipolar disorder, autoimmune diseases, chronic pain, asthma, depression, and other conditions, can be characterized by vectors (e.g., for bipolar disorder, the simplest vectors are feeling energized and feeling positive vs negative). The position of a user with that condition on these vectors may represent the person's current state. These vectors may have thresholds for ‘normal’ or ‘good’ states and/or ‘bad’ states. A position on a coordinate system may shift over time as the person's state shifts and the position may be used to identify patterns in mood and health, as well as to predict a direction which the user is moving (e.g., closer to, or further from, a “good” or “bad” state). By identifying what a particular user's thresholds are for “good” or “bad” states and where the user is on a curve or other health pattern, the functionality provided by the methods and systems described herein may activate components of the system that help direct the user towards ‘normal/good’ states on the vectors. This is in contrast to conventional tools, which do not typically provide users with means for identifying a location on a coordinate system that the user identifies as representing their current state, relying instead on paper-based lists of questions from which a human reviewer may deduce a user's state; such conventional tools may rely upon a standardized questionnaire (paper or digital) that does not require any assessment, but includes a simple system of points that are added to a total which represents a certain status of a patient.

The method includes accessing, by the application, from at least one data source, data associated with the user (104). The application 204, including one or more backend services, may receive data from, without limitation, sensors, medical integrations (e.g., electronic health records, including DNA analyses, lab data, in-home tests, and so on), and direct or indirect user input. Integration with user services may provide functionality for gathering of data from additional sources (including, without limitation, APPLE HEALTHKIT, GOOGLE FIT, 3rd party health gadget APIs, and 3rd party services). The application 204 may use the accessed data to generate advice, predictions, reports, alerts, and other information for the user from received data. The application 204 may include one or more machine learning and heuristics engines with which to analyze received data and for generating advice and predictions. The application 204 may interact with a platform 210 executed on a computing device 206 remotely located from the client computing device 202 and the platform 210 may execute one or more machine learning and heuristics engines with which to analyze received data and for generating advice and predictions. The application may store the received data from the one or more data sources in an encrypted data store.

As shown in FIG. 2C, the platform 210 may provide access to a plurality of services. The platform 210 may provide one or more other services in addition to machine learning and heuristics engines including, without limitation, web-based access to a user's data (e.g., a web app the user may interact with instead of or in addition to the application 204 executing on the user's client computing device 102), one or more interfaces to third party services (e.g., user services, data sources, one or more knowledge bases providing access to medical research, federated machine learning and/or natural language processing services, and/or partner services), and access to one or more cloud services. The platform 210 may have access to one or more electronic health records associated with the user (e.g., user medical data). The platform 210 may execute and populate its own electronic health record subsystem for maintaining one or more user's health data. As the platform 210 receives data for one or more users and executes to make recommendations and to analyze user data received subsequent to making recommendations, the platform 210 may receive additional data about how users health status changes (or remains the same) throughout a plurality of interactions with the system 200; the platform 210 may use this data to improve subsequent recommendations for all users. By way of example, the system 200 may receive data from sources such as electronic medical records, external data (e.g., weather and air quality and other environmental factors), user sensors, self-reported data from the user, and other data as described herein; the platform 210 may use this continuously received data to improve time series analyses, identification of health dynamics, and the execution of the machine learning models that select recommendations for users. The system 200 may therefore be said to learn from the connected data received.

As indicated in FIG. 2C, and as will be described in additional detail below, the application 204 may provide data to the platform 210 for stream processing; the platform 210 may include or be in communication with a stream processor for processing internet of things (IoT) data (e.g., sensor data) via a sink to a data-lake. The application 204 may transfer encrypted data to the stream processor and the data may remain encrypted as it is transferred to the data lake for storage.

The application 204 may include or have access to, customer data (including, without limitation, a profile of personal data, passively-sensed health data, optional community data, and medical data retrieved from, for examples, electronic health records databases or other sources of HL7/FHIR data). The application 204 may include application data, including a central platform interface and one or more health API interfaces. The application 204 may include one or more components accessing third party APIs to retrieve or transmit data.

The application 204 may comply with the HL7 standard for patient data interaction. The application 204 may comply with the FHIR standard for patient data interaction. The application 204 may comply with one or both of these standards. The application 204 may be designed to provide a level of privacy and security including, without limitation, one or more of the following: an architecture based on EHR industry standards; HIPAA & GDPR Compliance; FHIR Interconnectivity framework; pseudo anonymization with integrated mechanism for full anonymization on user request; user's full ownership and control of data; auditability prepared; encrypted data transmission and storage as well as encrypted backups; personal data pods fully controlled by the user; federated learning or similar mechanisms for distributed data analysis; full employee awareness of security and privacy by design; and/or implementation of solid processes to reduce data access to a need-to-only basis. The platform 210 may similarly comply with one or more standards for patient data interaction to provide a level of privacy and/or security.

In one embodiment, the application 204 gathers data from one or more wearables associated with a user of the computing device 202 and gathers data about an environment of the user, which presents a current situation pattern as well as, in combination with historical data, a situation evolution pattern. The application 204 may detect patterns which are suboptimal for the user's health state and provide behavioral recommendations to change these patterns for the better. The application 204 may leverage a number of systems or services to provide such functionality. The application 204 may, for example, be in communication with the platform 210. The application 204 may exchange data with the platform 210, which may provide additional resources for analyzing the accessed data and detecting patterns within the data relevant to the user's health state and behavioral patterns.

As one or more devices or services associated with the user generate data, the application 204 receives the generated data. For example, the user may generate data via one or more wearable devices (smartwatch, digital scale, airflow meter, fitness tracker, pedometers, etc.). The application 204 may receive authorization to access data generated by the user's computing device 202, such as metrics describing how much time the user spends on the computing device 202 and/or on specific applications or categories of applications executed by, or made available to the user by, the computing device 202. The application 204 may receive authorization to access data from the user's account(s) with one or more third party providers, for example, and without limitation, from social media accounts, online music accounts, calendaring data, computer programming accounts, bank accounts, credit card accounts, email accounts, and so on. The user's computing device may collect location data (mobility data, local weather, local air quality, etc.) and provide that data to the application 204. The computing device 202 may store the data (e.g., for APPLE devices in APPLE HEALTHKIT, for ANDROID devices in the user's personal GOOGLE cloud entity for GOOGLE FIT). The application 204 may receive the data when the user next executes the application.

The application 204 may use a stream processing infrastructure to capture the gathered and stored data; as will be understood by those of skill in the art, stream processing allows for transmitting and exchanging data in a high data density architecture. Stream processing may include collection of data from the user, mapping/reducing the data to logic, and generation of insights and recommendations by one or more machine learning services, which may be executed by either the client computing device 202, the computing device 206 on which the platform 210 executes or both.

The application 204 may store reduced sets of the data in a personalized record in a secure electronic health record provider application, such as, without limitation, AIDBOX, hosted on a server maintained by an entity providing the application to the user such as the computing device 206 (e.g., hosted on one or more controlled Kubernetes instances). Alternatively, a separate computing device than the computing device 206 that hosts the platform 210 may provide the storage for the reduced data sets. The system may include a database 220 for such data storage.

In one embodiment, the application 204 may store received data anonymized or pseudo-anonymized in a data-lake or other centralized repository of data stored in its natural/raw format, usually object blobs or files, which may be structured or unstructured. The reduction of data allows for storing certain data properties within personal accounts (hence not necessarily anonymous); the stored properties may be determined by applying one or more rules to identify data deemed “relevant” (e.g., identifying certain types or structures of data and/or data from certain data sources and/or data predetermined to be used in further processing steps). The reduction logic may be based on one or more machine learning engines, which may specify a level of data-depth for data to retain in order for the data to remain “meaningful” or “useful” in further processing steps by the machine learning engine.

The method includes analyzing, by the application, the accessed data and the received user input (106). Having received access to the data, the application 204 may analyze the data. The application 204 may use one or more heuristics tools and/or machine learning tools to match the analyzed data with one or more known health patterns. The application 204 may transmit the data or a subset of the data to the platform 210 for analysis.

The method includes identifying, by the application, a health pattern of the user based on the analyses (108). The application 204 may provide the user with an interpretation of the one or more health patterns detected. The application 204 may identify behaviors which could have a positive impact on the detected health pattern. The application 204 may match the identified behaviors with a situation of the user. The application 204 may match the identified behaviors with one or more preferences known from the user (including, without limitations, preferences explicitly identified by the user—such as in response to a question displayed in a user interface 208 of the application 204 or by preferences inferred from analyzing data gathered about the user). In one embodiment, the application 204 applies one or more heuristics tools to perform the matching. In another embodiment, the application 204 leverages one or more machine learning engines to execute one or more recommendation algorithms. By learning both the individual effects (e.g., adherence, health gain, etc.) as well as the general effects (e.g., impact on health in distinct cohorts), the application 204 may generate data that the system 200 may provide to a machine learning model (e.g., either on the client computing device 202 or on the computing device 206 and/or in communication with the platform 210) executed to improve heuristics-based recommendations, including recommendations regarding the time and location of recommending certain behaviors, behaviors based on circumstances, based on health situation, etc.

As shown in FIG. 2A, the system 200 may include one or more machine learning engines 212a-n. The client computing device 202 may execute a machine learning engine 212. The platform 210 may execute or be in communication with a machine learning engine 212. Similarly, the system 200 may include one or more recommendation engines 214a-n. The client computing device 202 may execute a recommendation engine 214. The platform 210 may execute or be in communication with a recommendation engine 214. The machine learning engine 212 and the recommendation engine 214 may each provide more functionality on one device than the other—for example, a user may instruct the system to provide a limited set of functionality and only execute the machine learning engine and/or recommendation engines on the client computing device 202 to increase security by minimizing the number of computing device with access to user data; alternatively, another user may instruct the system to provide an increased set of functionality by allowing the platform 210 to access the user data and to provide additional computing resources (e.g., additional machine learning engines and/or recommendation engines).

In an embodiment in which the platform 210 executes the recommendation engine 214, the application 204 may interact with a recommendation engine Application Programming Interface (API) to exchange data with the recommendation engine 214. The recommendation engine 214 may include or be in communication with a recommendation database (not shown). the recommendation engine 214 may apply one or more filters to each of a plurality of recommendations in the recommendation database, the filters selected based upon data provided by the application 204 and associated with a user for which a recommendation is being selected. The recommendation engine 214 may transmit one or more recommendations to the application 204 via the recommendation engine API. As will be described in further detail, prior to transmitting the one or more recommendations to the application 204, the recommendation engine 214 may communicate with one or more additional engines of the platform 210, such as a user behavior preference model engine, a behavior selection engine, and/or any other engines executed on the platform 210 including those shown in FIGS. 2D and 2E.

The method includes determining, by the application, whether the health pattern of the user is aligned with a goal of the user (110). Whether the client computing device 202 or the platform 210 execute the recommendation engine 214, recommendations for the user may be based on health goals and whether the health pattern is aligned with one or more health goals, e.g., increase heart rate variability (HRV), increase mobility, decrease behavior X, increase vitamin Y level, avoid XYZ; recommendations may also specify a measure to quantify how to reach a health goal. For instance, for a user who enjoys going to museums and who has a goal of walking 10,000 steps a day, recommendations may include recommendation for types of activities and provide examples as to how the activities will help achieve the goal, such as going to a museum, which includes walking to museum, 1500 steps; walking in the museum, 4000 steps; walking back, 1500 steps; goal contribution: 70%/7000 steps. In this way, a recommendation engine of the application 204 may select activities and combinations of activities to recommend to a particular user.

The method includes generating, by the application, a recommendation to the user to align at least one user behavior pattern with the goal of the user, the recommendations personalized to a preference of the user and having a level of effectiveness satisfying a threshold level of effectiveness (112). The application 204 may make one or more recommendations to the user. The application 204 may track a user's progress and quantifies the progress (e.g., how a user's self-identified mood and energy change over time, or fail to change, as the user does or does not implement a recommendation). In one embodiment, the application 204 may track user activity and after a certain amount of time, the application 204 will know how the user activity has changed over time. In another embodiment, the application 204 may display one or more questions to a user, such as, without limitation, asking how much time the user spent exercising. In still another embodiment, the application 204 may receive data from the computing device or from one or more external devices to determine heart rate variability, resting heart rate, weight, muscle mass vs bone, fat and water mass, etc., for use as a basis to estimate a level of fitness. The application 204 may make updates to its models (effectiveness, personal preferences, etc.) subsequent to analyzing and quantifying user progress.

As indicated above, the system 200 may include, or provide access to, a recommendation engine 214. Referring now to FIG. 2D, a block diagram depicts one embodiment of the system 200 in which the platform 210 executes one or more engines to process user input. As shown in FIG. 2D, the platform 210 may include, or be in communication with, a behavior recommendation engine, a user behavior preference model engine, a behavior selection engine, a condition state model engine, a user condition analytics engine, and an interpretation formulation engine. The platform 210 may execute one or more of these components to identify one or more health patterns of the user as indicated above in connection with FIG. 1, (108). The platform 210 may further execute one or more of the engines shown in FIG. 2D to assess a health state of the user, identify one or more personal preferences of the user, select at least one recommendation (e.g., from a database of recommendations), and analyze whether the recommendations were implemented by the user and whether doing so improved an aspect of the user's wellbeing (e.g., by receiving subsequent user input to the user interface or to sensor data tracking one or more aspects of the user's health). Recommendations are driven by the analysis of the received data by the recommendation engine 214.

As shown in FIG. 2E, a block diagram depicts one embodiment of the system 200 in which the platform 210 executes one or more user-focused services and one or more data science engines. As shown in FIG. 2E, the user-focused services may include, without limitation, a behavior recommendation engine, a user behavior preference model engine, a behavior selection engine, a user condition analytics engine, a condition state model engine, and an interpretation formulation engine. As shown in FIG. 2E, the data science back end services may include, without limitation, a behavior effectiveness analysis engine, and a condition state model analysis engine. The platform 210 may execute one or more of these components to identify one or more health patterns of the user as indicated above in connection with FIG. 1, (108). The back end services may receive requests from one or more sub-processes (e.g., from one or more of the user-focused services) and execute one or more of the engines to identify a recommendation that would impact at least one aspect of the user's health state and satisfy a threshold level of likelihood of implementation by the user. The platform 210 may execute one or more of these components to analyze subsequently received data and determine whether the user implemented the recommendation and whether the user's decision regarding whether or not to implement the recommendation impacted the user's health.

By way of example, and without limitation, the behavior recommendation engine shown in FIG. 2E may receive user data, extract a subset of the data, analyze the subset and/or one or more attributes or characteristics of the data (e.g., determining how many steps the user walked in a day for each of a number of days and determine an average number of steps the user walked in a period of time and determine that the user's step count for the period is similar to users who are categorized as one of a plurality of types of user—for example, moderately active users—and determine (e.g., by accessing a mapping or table or other data structure) that moderately active users are associated with a plurality of types of recommendations. Continuing with this example, the behavior recommendation engine may communicate with the behavior selection engine to determine which of the plurality of types of recommendations for this particular user are likely to meet or exceed a threshold level of likelihood of implementation—for example, the behavior selection engine may communicate with the user behavior preference model engine to determine which of the available recommendations satisfy one or more user preferences, such as, without limitation, not running on a treadmill or not exercising outside when the temperature is outside of a specified range. The behavior recommendation engine may communicate with the behavior effectiveness analysis engine to provide an update as to what recommendation was made so that then the behavior effectiveness analysis engine receives subsequent user data associated with recommendations implemented (or not) and health or emotional status, the behavior recommendation engine can determine a level of effectiveness of the recommendation for the user—and, in some embodiments, to determine a level of effectiveness for users having at least one characteristic in common with the specific user whose data is analyzed (e.g., moderately active users or users who received a particular recommendation).

In one embodiment, the recommendation addresses two axes: effectiveness and personalization. The first is an effectiveness axis, e.g., what behavioral change will help someone with condition X with values XYZ and feeling X. The second axis is the personalization axis, e.g., what would person X like to do in situation Y, if their hobbies are XYZ, their interests are XYZ, they have time at X, they are in location Y, they have budget of Z. The more information the application 204 receives, the less general the system 200 needs to be in the formulation of the recommendations. The more the application 204 knows about the effectiveness of the recommendations, the better the recommendation engine 214 can assess the type of recommendation to provide. In one embodiment, “effectiveness” as used herein refers to a user's reported improvement. For depression, for example, this may be reported through the mood check, where better mood or less deterioration of mood for a certain time means improvement. For asthma, as another example, the application may ask about shortness of breath, emergency inhaler intake, etc., and less of these during a certain amount of time may mean improvement. For bi-polar measuring, as a further example, the more stable a person is in their mood interpretation and energy interpretation, and the less negative their mood, the better they are, which is another signal of improvement. Furthermore, certain cues may provide an indication of wellness; for instance, without limitation, it is known that those with bi-polar disorders that are in their high moods may have erratic spending patterns, go on shopping sprees, and so on, whereas people with depression are known to spend more time on their mobile phones.

Once the recommendation engine 214 has analyzed received data, the recommendation engine may identify a category of recommendations to select from, based on one or more attributes of the received data identified during the analysis. Recommendations may be formulated from very broad to very narrow based on what data the system 200 has about the user. For instance, the recommendation engine 214 may recommend that someone should be more physically active, or, the recommendation engine 214 may tell them to go for a 10 minute walk, or, the recommendation engine 214 may recommend that they do their favorite sports, or if they like movies and live in city X, the recommendation may tell them about movie Y playing in a cinema at walking distance, but if it is raining, and they like art, the recommendation engine 214 may tell them about a museum which they can reach by public transport. Therefore, in some embodiments, a recommendation includes identifiers of one or more activities which may include one or more of the following: goal parameter attributes and values (which may also be negative, for instance if an activity is outdoors and the air quality is bad, there is negative effect on the goal of breathing fresh air), attributes of user interests and/or hobbies attributes, user budget attributes, spatial attributes (location, in door, outside, time of year, type of weather), combination requirements (a goal to go to a museum will net to be combined with getting there).

The recommendation engine 214 may include, or have access to, functionality for providing variety in the selection of recommendations (e.g., not providing the same or similar recommendations each time the application provides a recommendation). The recommendation engine 214 may also vary on type of recommendation (physical, social, cultural, etc.). The recommendation engine 214 may also provide the functionality to determine that some users prefer stability and structure, in which case they may prefer having similar recommendations at a fixed interval, and the recommendation engine 214 may make recommendations accordingly. Similarly, the recommendation engine 214 may provide functionality to determine that some users prefer variety, some people have very limited interests and will not want variety, and so on, and take into account such traits in fine tuning personalized recommendations for each type of user.

To provide more accurate recommendations, the recommendation engine 214 may be trained along one or more dimensions. The recommendation engine 214 may be trained to identify which recommendations are effective, which the application 204 may do on a personal level, e.g., identifying whether a user's scores improve, as well as on a population level, e.g., identifying which recommendations have best score gains across the population of users. The recommendation engine 214 may be trained to identify which recommendation fits a user (e.g., satisfying one or more user preferences), which the application 204 may do on a personal level, using feedback mechanisms to identify better and worse recommendation, as well as on a population level, which recommendations resonate well with people with similar attributes. The recommendation engine 214 may be trained to leverage a data model that is itself trained to identify recommendations in a plurality of recommendations that would match a user's situation and preferences (and, in some embodiments, that would satisfy a threshold level of matching).

As shown in FIG. 2F, the system 200 may provide one or more APIs for communication between one or more components. By way of example, the application 204 may use a user API to provide IoT data for stream processing and the stream processor may use a machine learning model API proxy to provide data to one or more machine learning processes (including, without limitation, the engines shown in FIGS. 2D and 2E, as well as the data science models shown in FIG. 2F). In embodiments in which the system 200 includes a proprietary electronic health records system, the system 200 may include an interoperability API for accessing data stored by that EHR system. Components in the system may use a Services API to access databases provided by the system 200 such as databases storing specific types of recommendations, insights, and feedback to provide to users.

The method includes modifying, by the application, the user interface to display the recommendation (114). The application 204 may provide the user with recommendations received from the recommendation engine 214 (e.g., by dynamically updating the user interface 208 to include a display of text, image, video, audio, or other content accessible to a user and representing the recommendation).

The platform 210 may include, or be in communication with, an interpretation engine 216. The interpretation engine 216 may generate interpretations of data from the user and their environment as well as making interpretations about what could improve the user environment. Interpretations may have one or more levels. One such level is a daily interpretation, such as “Today is an especially bad day for asthma as it is humid, air quality is low and there are lots of pollen in the air”. A second example of an interpretation is a time-series interpretation, such as “I see that you have been feeling worse the last couple of weeks; you also sleep less and are less active”. Another example is a population interpretation, such as “we see that people feel better when they do exercises at least 3 times a week” or “we see that people with asthma feel worse in autumn.”

The platform 210 may include, or have access to, a conversation engine 218 to serve a plurality of functions. The conversation engine 218 may generate and display an interface with which to gather information, including personal information, information about preferences, activities, hobbies, personal/professional/family situation, availability, budget, condition state, feedback. The conversation engine 218 may also generate and display an interface to provide behavioral feedback, as well as providing an interface to present findings and interpretations.

By allowing a user to chat with the application 204 via an interface provided by the conversation engine 218, the system 200 provides functionality with which the application 204 may present one or more daily questions and present other questionnaires at regular intervals. The application 204 may also request open text input that may be saved in a journal associated with the user. The chat interface may also provide functionality for gathering personal information about the user's preferences. The chat engine 218 may generate one or more questions that the application 204 may display to the user. Examples of such questions include, without limitation: “What do you like to do on a Sunday?” “How does a normal week day look like for you?” “How would a perfect day look like?” “Who do you meet regularly?” “Are you interested in art/sports/culture?” “Which arts do you like?”, etc. By way of example, and without limitation, the conversation engine 218 may provide AI-enabled chat functionality leveraging one or more third party chatbot services.

The application 204 may also be able to make recommendations (e.g., from one or more recommendation engines) and ask for feedback through the chat interface populated by the conversation engine 218; for example: “Hi Joe, it is Saturday, the weather is great, would you like to go for a walk today?” “Why not?” “Which of these other activities would be more your thing?” Also, interpretations can be provided via the chat interface, such as “Today is an awful day for people suffering asthma, bad air quality, humid, many pollen.”

The system 200 may include a goal engine 220 providing functionality for specifying, recommending, and/or modifying goals. From the recommendations generated, the application 204 may support the user in implementing one or more of the recommendations; the goals engine 220 may provide functionality for doing so. As an example, without limitation the goals engine 220 may connect a user to a reward system, show the user progress towards a goal, and otherwise support the user in achieving one or more goals. In one embodiment, users may manually specify one or more goals or choose one or more goals from a list of options.

Therefore, the methods and systems described herein provide functionality for generating personalized recommendations and predictions of a level of effectiveness of the personalized recommendation for a type of user. By identifying factors and thresholds specific to a particular user, or type of user, the systems described herein may generate and provide personalized recommendations that guide the user in a certain behavioral direction, leveraging machine learning to identify dimensions of behavior including personalization and effectiveness to increase the likelihood that the user will implement effective recommendations and to increase the likelihood that the application will influence the user's behavior. Analyzing the user's data—which may include, without limitation, input identifying a current state of wellbeing (e.g., via a user interface 208 displaying a quadrant from which the user may identify a portion that represents the user's level of wellbeing)—allows the system to identify where a user is in a health pattern and what direction the user is moving towards, as well as to identify personalized and effective behaviors that the user can implement to move their health patterns in a direction in line with a user's stated goals.

The methods and systems described herein may provide functionality for analyzing an identification of a first level of wellbeing for a user at a first time and, based upon the analysis, predicting a second level of wellbeing for the user at a subsequent time. Such functionality may serve as an “early warning system” able to alert one or more users to a particular user's condition and a possibility for a subsequent worsening of that condition.

The methods and systems described herein may provide functionality for comparing data associated with one user with population-level data. By applying patterns identified from a larger sample to the data provided by or received about a user, such functionality may generate improved recommendations for the user.

In some embodiments, the system 200 includes non-transitory, computer-readable medium comprising computer program instructions tangibly stored on the non-transitory computer-readable medium, wherein the instructions are executable by at least one processor to perform each of the steps described above in connection with FIG. 1.

It should be understood that the systems described above may provide multiple ones of any or each of those components and these components may be provided on either a standalone machine or, in some embodiments, on multiple machines in a distributed system. The phrases ‘in one embodiment,’ ‘in another embodiment,’ and the like, generally mean that the particular feature, structure, step, or characteristic following the phrase is included in at least one embodiment of the present disclosure and may be included in more than one embodiment of the present disclosure. Such phrases may, but do not necessarily, refer to the same embodiment. However, the scope of protection is defined by the appended claims; the embodiments mentioned herein provide examples.

The terms “A or B”, “at least one of A and/or B”, “at least one of A and B”, “at least one of A or B”, or “one or more of A and/or B” used in the various embodiments of the present disclosure include any and all combinations of words enumerated with it. For example, “A or B”, “at least one of A and B” or “at least one of A or B” may mean (1) including at least one A, (2) including at least one B, (3) including either A or B, or (4) including both at least one A and at least one B.

The systems and methods described above may be implemented as a method, apparatus, or article of manufacture using programming and/or engineering techniques to produce software, firmware, hardware, or any combination thereof. The techniques described above may be implemented in one or more computer programs executing on a programmable computer including a processor, a storage medium readable by the processor (including, for example, volatile and non-volatile memory and/or storage elements), at least one input device, and at least one output device. Program code may be applied to input entered using the input device to perform the functions described and to generate output. The output may be provided to one or more output devices.

Each computer program within the scope of the claims below may be implemented in any programming language, such as assembly language, machine language, a high-level procedural programming language, or an object-oriented programming language. The programming language may, for example, be LISP, PROLOG, PERL, C, C++, C#, JAVA, Python, Rust, Go, or any compiled or interpreted programming language.

Each such computer program may be implemented in a computer program product tangibly embodied in a machine-readable storage device for execution by a computer processor. Method steps may be performed by a computer processor executing a program tangibly embodied on a computer-readable medium to perform functions of the methods and systems described herein by operating on input and generating output. Suitable processors include, by way of example, both general and special purpose microprocessors. Generally, the processor receives instructions and data from a read-only memory and/or a random access memory. Storage devices suitable for tangibly embodying computer program instructions include, for example, all forms of computer-readable devices, firmware, programmable logic, hardware (e.g., integrated circuit chip; electronic devices; a computer-readable non-volatile storage unit; non-volatile memory, such as semiconductor memory devices, including EPROM, EEPROM, and flash memory devices; magnetic disks such as internal hard disks and removable disks; magneto-optical disks; and CD-ROMs). Any of the foregoing may be supplemented by, or incorporated in, specially-designed ASICs (application-specific integrated circuits) or FPGAs (Field-Programmable Gate Arrays). A computer can generally also receive programs and data from a storage medium such as an internal disk (not shown) or a removable disk. These elements will also be found in a conventional desktop or workstation computer as well as other computers suitable for executing computer programs implementing the methods described herein, which may be used in conjunction with any digital print engine or marking engine, display monitor, or other raster output device capable of producing color or grayscale pixels on paper, film, display screen, or other output medium. A computer may also receive programs and data (including, for example, instructions for storage on non-transitory computer-readable media) from a second computer providing access to the programs via a network transmission line, wireless transmission media, signals propagating through space, radio waves, infrared signals, etc.

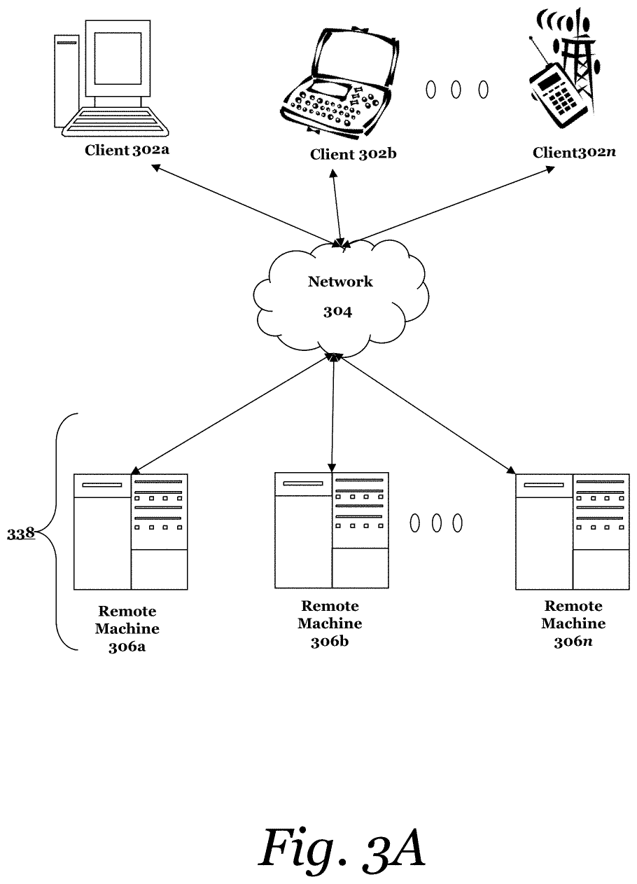

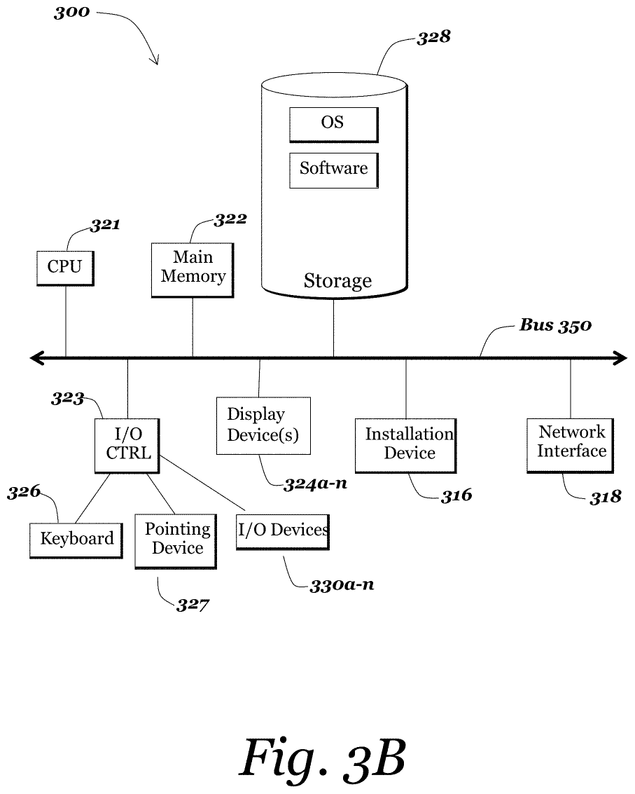

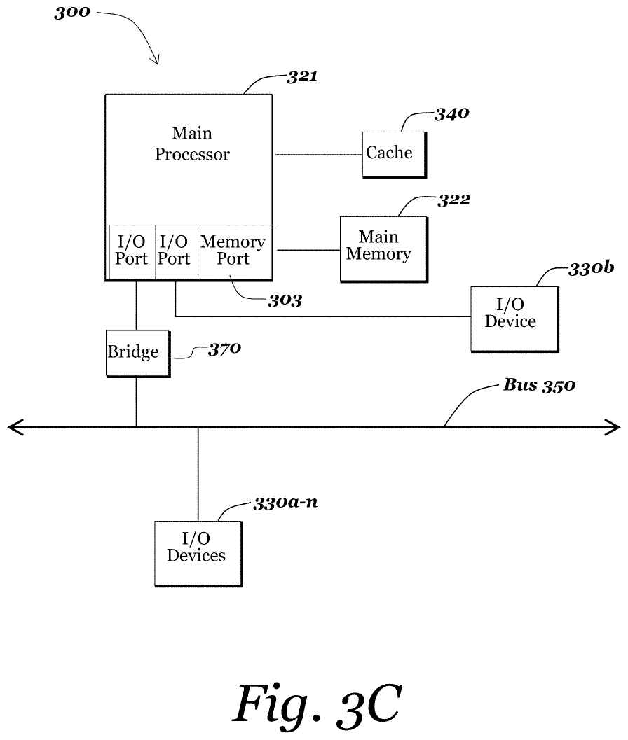

Referring now to FIGS. 3A, 3B, and 3C, block diagrams depict additional detail regarding computing devices that may be modified to execute novel, non-obvious functionality for implementing the methods and systems described above.

Referring now to FIG. 3A, an embodiment of a network environment is depicted. In brief overview, the network environment comprises one or more clients 302a-302n (also generally referred to as local machine(s) 302, client(s) 302, client node(s) 302, client machine(s) 302, client computer(s) 302, client device(s) 302, computing device(s) 302, endpoint(s) 302, or endpoint node(s) 302) in communication with one or more remote machines 306a-306n (also generally referred to as server(s) 306 or computing device(s) 306) via one or more networks 304.

Although FIG. 3A shows a network 304 between the clients 302 and the remote machines 306, the clients 302 and the remote machines 306 may be on the same network 304. The network 304 can be a local area network (LAN), such as a company Intranet, a metropolitan area network (MAN), or a wide area network (WAN), such as the Internet or the World Wide Web. In some embodiments, there are multiple networks 304 between the clients 302 and the remote machines 306. In one of these embodiments, a network 304′ (not shown) may be a private network and a network 304 may be a public network. In another of these embodiments, a network 304 may be a private network and a network 304′ a public network. In still another embodiment, networks 304 and 304′ may both be private networks. In yet another embodiment, networks 304 and 304′ may both be public networks.

The network 304 may be any type and/or form of network and may include any of the following: a point to point network, a broadcast network, a wide area network, a local area network, a telecommunications network, a data communication network, a computer network, an ATM (Asynchronous Transfer Mode) network, a SONET (Synchronous Optical Network) network, an SDH (Synchronous Digital Hierarchy) network, a wireless network, a wireline network, an Ethernet, a virtual private network (VPN), a software-defined network (SDN), a network within the cloud such as AWS VPC (Virtual Private Cloud) network or Azure Virtual Network (VNet), and a RDMA (Remote Direct Memory Access) network. In some embodiments, the network 304 may comprise a wireless link, such as an infrared channel or satellite band. The topology of the network 304 may be a bus, star, or ring network topology. The network 304 may be of any such network topology as known to those ordinarily skilled in the art capable of supporting the operations described herein. The network may comprise mobile telephone networks utilizing any protocol or protocols used to communicate among mobile devices (including tables and handheld devices generally), including AMPS, TDMA, CDMA, GSM, GPRS, UMTS, or LTE. In some embodiments, different types of data may be transmitted via different protocols. In other embodiments, the same types of data may be transmitted via different protocols.

A client 302 and a remote machine 306 (referred to generally as computing devices 300 or as machines 300) can be any workstation, desktop computer, laptop or notebook computer, server, portable computer, mobile telephone, mobile smartphone, or other portable telecommunication device, media playing device, a gaming system, mobile computing device, or any other type and/or form of computing, telecommunications or media device that is capable of communicating on any type and form of network and that has sufficient processor power and memory capacity to perform the operations described herein. A client 302 may execute, operate or otherwise provide an application, which can be any type and/or form of software, program, or executable instructions, including, without limitation, any type and/or form of web browser, web-based client, client-server application, an ActiveX control, a JAVA applet, a webserver, a database, an HPC (high performance computing) application, a data processing application, or any other type and/or form of executable instructions capable of executing on client 302.

In one embodiment, a computing device 306 provides functionality of a web server. The web server may be any type of web server, including web servers that are open-source web servers, web servers that execute proprietary software, and cloud-based web servers where a third party hosts the hardware executing the functionality of the web server. In some embodiments, a web server 306 comprises an open-source web server, such as the APACHE servers maintained by the Apache Software Foundation of Delaware. In other embodiments, the web server executes proprietary software, such as the INTERNET INFORMATION SERVICES products provided by Microsoft Corporation of Redmond, Wash., the ORACLE IPLANET web server products provided by Oracle Corporation of Redwood Shores, Calif., or the ORACLE WEBLOGIC products provided by Oracle Corporation of Redwood Shores, Calif.

In some embodiments, the system may include multiple, logically-grouped remote machines 306. In one of these embodiments, the logical group of remote machines may be referred to as a server farm 338. In another of these embodiments, the server farm 338 may be administered as a single entity.

FIGS. 3B and 3C depict block diagrams of a computing device 300 useful for practicing an embodiment of the client 302 or a remote machine 306. As shown in FIGS. 3B and 3C, each computing device 300 includes a central processing unit 321, and a main memory unit 322. As shown in FIG. 3B, a computing device 300 may include a storage device 328, an installation device 316, a network interface 318, an I/O controller 323, display devices 324a-n, a keyboard 326, a pointing device 327, such as a mouse, and one or more other I/O devices 330a-n. The storage device 328 may include, without limitation, an operating system and software. As shown in FIG. 3C, each computing device 300 may also include additional optional elements, such as a memory port 303, a bridge 370, one or more input/output devices 330a-n (generally referred to using reference numeral 330), and a cache memory 340 in communication with the central processing unit 321.

The central processing unit 321 is any logic circuitry that responds to and processes instructions fetched from the main memory unit 322. In many embodiments, the central processing unit 321 is provided by a microprocessor unit, such as: those manufactured by Intel Corporation of Mountain View, Calif.; those manufactured by Motorola Corporation of Schaumburg, Ill.; those manufactured by Transmeta Corporation of Santa Clara, Calif.; those manufactured by International Business Machines of White Plains, N.Y.; or those manufactured by Advanced Micro Devices of Sunnyvale, Calif. Other examples include RISC-V processors, SPARC processors, ARM processors, and processors for mobile devices. The computing device 300 may be based on any of these processors, or any other processor capable of operating as described herein.

Main memory unit 322 may be one or more memory chips capable of storing data and allowing any storage location to be directly accessed by the microprocessor 321. The main memory 322 may be based on any available memory chips capable of operating as described herein. In the embodiment shown in FIG. 3B, the processor 321 communicates with main memory 322 via a system bus 350. FIG. 3C depicts an embodiment of a computing device 300 in which the processor communicates directly with main memory 322 via a memory port 303. FIG. 3C also depicts an embodiment in which the main processor 321 communicates directly with cache memory 340 via a secondary bus, sometimes referred to as a backside bus. In other embodiments, the main processor 321 communicates with cache memory 340 using the system bus 350.

In the embodiment shown in FIG. 3B, the processor 321 communicates with various I/O devices 330 via a local system bus 350. Various buses may be used to connect the central processing unit 321 to any of the I/O devices 330, including a VESA VL bus, an ISA bus, an EISA bus, a MicroChannel Architecture (MCA) bus, a PCI bus, a PCI-X bus, a PCI-Express bus, or a NuBus. For embodiments in which the I/O device is a video display 324, the processor 321 may use an Advanced Graphics Port (AGP) to communicate with the display 324. FIG. 3C depicts an embodiment of a computing device 300 in which the main processor 321 also communicates directly with an I/O device 330b via, for example, HYPERTRANSPORT, RAPIDIO, or INFINIBAND communications technology.

One or more of a wide variety of I/O devices 330a-n may be present in or connected to the computing device 300, each of which may be of the same or different type and/or form. Input devices include keyboards, mice, trackpads, trackballs, microphones, scanners, cameras, and drawing tablets. Output devices include video displays, speakers, inkjet printers, laser printers, 3D printers, and dye-sublimation printers. The I/O devices may be controlled by an I/O controller 323 as shown in FIG. 3B. Furthermore, an I/O device may also provide storage and/or an installation medium 316 for the computing device 300. In some embodiments, the computing device 300 may provide USB connections (not shown) to receive handheld USB storage devices such as the USB Flash Drive line of devices manufactured by Twintech Industry, Inc. of Los Alamitos, Calif.

Referring still to FIG. 3B, the computing device 300 may support any suitable installation device 316, such as a floppy disk drive for receiving floppy disks such as 3.5-inch, 5.25-inch disks or ZIP disks; a CD-ROM drive; a CD-R/RW drive; a DVD-ROM drive; tape drives of various formats; a USB device; a hard-drive or any other device suitable for installing software and programs. In some embodiments, the computing device 300 may provide functionality for installing software over a network 304. The computing device 300 may further comprise a storage device, such as one or more hard disk drives or redundant arrays of independent disks, for storing an operating system and other software. Alternatively, the computing device 300 may rely on memory chips for storage instead of hard disks.

Furthermore, the computing device 300 may include a network interface 318 to interface to the network 304 through a variety of connections including, but not limited to, standard telephone lines, LAN or WAN links (e.g., 802.11, T1, T3, 56 kb, X.25, SNA, DECNET, RDMA), broadband connections (e.g., ISDN, Frame Relay, ATM, Gigabit Ethernet, Ethernet-over-SONET), wireless connections, virtual private network (VPN) connections, or some combination of any or all of the above. Connections can be established using a variety of communication protocols (e.g., TCP/IP, IPX, SPX, NetBIOS, Ethernet, ARCNET, SONET, SDH, Fiber Distributed Data Interface (FDDI), RS232, IEEE 802.11, IEEE 802.11a, IEEE 802.11b, IEEE 802.11g, IEEE 802.11n, 802.15.4, Bluetooth, ZIGBEE, CDMA, GSM, WiMax, and direct asynchronous connections). In one embodiment, the computing device 300 communicates with other computing devices 300′ via any type and/or form of gateway or tunneling protocol such as GRE, VXLAN, IPIP, SIT, ip6tnl, VTI and VTI6, IP6GRE, FOU, GUE, GENEVE, ERSPAN, Secure Socket Layer (SSL) or Transport Layer Security (TLS). The network interface 318 may comprise a built-in network adapter, network interface card, PCMCIA network card, card bus network adapter, wireless network adapter, USB network adapter, modem, or any other device suitable for interfacing the computing device 300 to any type of network capable of communication and performing the operations described herein.

In further embodiments, an I/O device 330 may be a bridge between the system bus 350 and an external communication bus, such as a USB bus, an Apple Desktop Bus, an RS-232 serial connection, a SCSI bus, a FireWire bus, a FireWire 800 bus, an Ethernet bus, an AppleTalk bus, a Gigabit Ethernet bus, an Asynchronous Transfer Mode bus, a HIPPI bus, a Super HIPPI bus, a SerialPlus bus, a SCI/LAMP bus, a FibreChannel bus, or a Serial Attached small computer system interface bus.

A computing device 300 of the sort depicted in FIGS. 3B and 3C typically operates under the control of operating systems, which control scheduling of tasks and access to system resources. The computing device 300 can be running any operating system such as any of the versions of the MICROSOFT WINDOWS operating systems, the different releases of the UNIX and LINUX operating systems, any version of the MAC OS for Macintosh computers, any embedded operating system, any real-time operating system, any open source operating system, any proprietary operating system, any operating systems for mobile computing devices, or any other operating system capable of running on the computing device and performing the operations described herein. Typical operating systems include, but are not limited to: WINDOWS 3.x, WINDOWS 95, WINDOWS 98, WINDOWS 2000, WINDOWS NT 3.51, WINDOWS NT 4.0, WINDOWS CE, WINDOWS XP, WINDOWS 7, WINDOWS 8, WINDOWS VISTA, and WINDOWS 10 all of which are manufactured by Microsoft Corporation of Redmond, Wash.; MAC OS manufactured by Apple Inc. of Cupertino, Calif.; OS/2 manufactured by International Business Machines of Armonk, N.Y.; Red Hat Enterprise Linux, a Linux-variant operating system distributed by Red Hat, Inc., of Raleigh, N.C.; Ubuntu, a freely-available operating system distributed by Canonical Ltd. of London, England; CentOS, a freely-available operating system distributed by the centos.org community; SUSE Linux, a freely-available operating system distributed by SUSE, or any type and/or form of a Unix operating system, among others.

Having described certain embodiments of methods and systems for generating personalized recommendations and predictions of a level of effectiveness of the personalized recommendations for a user, it will be apparent to one of skill in the art that other embodiments incorporating the concepts of the disclosure may be used.

What is claimed is:

1. A method for generating at least one personalized recommendation and a prediction of a level of effectiveness of the personalized recommendation for a user, may comprise:
receiving, by an application executing on a computing device, via a user interface, user input identifying a portion of a quadrant displayed within the user interface descriptive of an emotional state of a user and of an energetic state of a user;
accessing, by the application, from at least one data source, data associated with the user;
analyzing, by the application, the accessed data and the received user input;
identifying, by the application, a health pattern of the user based on the analyses;
determining, by the application, whether the health pattern of the user is aligned with a goal of the user;
generating, by the application, a recommendation to the user to align at least one user behavior pattern with the goal of the user, the recommendations personalized to a preference of the user and having a level of effectiveness satisfying a threshold level of effectiveness; and
modifying, by the application, the user interface to display the recommendation.
